(19) United States
(12) Patent Application Publication
(10) Pub. No.: US 2023/0404971 A1
(43) Pub. Date: Dec. 21, 2023

(54) Combination Comprising Abemaciclib and 6-(2,4-Dichlorophenyl)-5-[4-[(3S)-1-(3-Fluoropropyl)Pyrrolidin-3-yl]Oxyphenyl]-8,9-Dihydro-7H-Benzo[7]Annulene-2-Carboxylic Acid

(71) Applicant: Sanofi, Paris (FR)

(72) Inventors: Monsif Bouaboula, Bridgewater, NJ (US); Zhuyan Guo, Bridgewater, NJ (US); Fangxian Sun, Bridgewater, NJ (US)

(73) Assignee: Sanofi, Paris (FR)

(21) Appl. No.: 18/037,949

(22) PCT Filed: Nov. 23, 2021
(86) PCT No.: PCT/EP2021/082583

§ 371 Date: May 19, 2023

(30) Foreign Application Priority Data
Nov. 23, 2020 (EP) 20315465.3

(51) Int. Cl. A61K 31/4015; A61K 31/506; A61P 35/00

(52) U.S. Cl. CPC A61K 31/4015; A61P 35/00; A61K 31/506

(57) ABSTRACT

Herein are provided a combination of abemaciclib and of 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, a pharmaceutical composition containing such a combination, and the therapeutic uses thereof, in particular for the treatment of cancer, including breast cancer.

Herein are provided a combination of abemaciclib and of 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, a pharmaceutical composition containing such combination, and the therapeutic uses of such combination and pharmaceutical composition, in particular for the treatment of cancer.

The estrogen receptor α (ESR1) is expressed in the majority of breast tumors, enabling them to respond to the mitogenic actions of estrogens.

6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, hereafter designated as “compound (1)”, is a selective estrogen receptor degrader (SERD) which has complete estrogen receptor antagonist properties and accelerates the proteasomal degradation of the estrogen receptor. This compound is disclosed in the patent application PCT/EP2017/053282, published as WO 2017/140669:

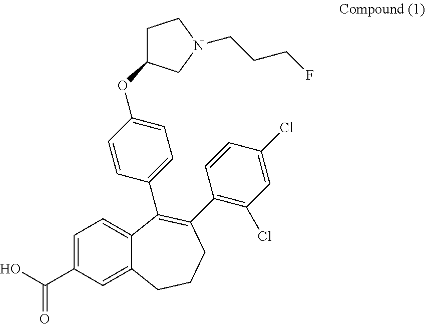 [SMILES: O=C(O)c1ccc2c(c1)CCCC(c1ccc(Cl)cc1Cl)=C2c1ccc(O[C@H]2CCN(CCCF)C2)cc1]

Abemaciclib, also known as N-{5-[(4-ethylpiperazin-1-yl)methyl]pyridin-2-yl}-5-fluoro-4-[4-fluoro-2-methyl-1-(propan-2- yl)-1H-benzimidazol-6-yl]pyrimidin-2-amine, is a kinase inhibitor, more specifically an inhibitor of CDK 4 and 6 (also called a “CDK4/6” inhibitor). It has the following formula:

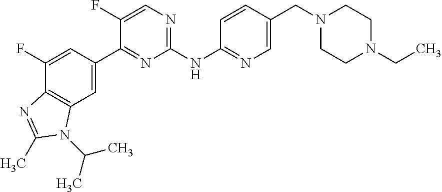 [SMILES: CCN1CCN(Cc2ccc(Nc3ncc(F)c(-c4cc(F)c5nc(C)n(C(C)C)c5c4)n3)nc2)CC1]

Abemaciclib is marketed, with VERNEZIO® as one of its tradenames. It is indicated as monotherapy for the treatment of adult patients with hormone receptor (HR)-positive, human epidermal growth factor receptor 2 (HER2)-negative advanced or metastatic breast cancer with disease progression following endocrine therapy and prior chemotherapy in the metastatic setting. It is also indicated in combination with the endocrine therapy fulvestrant for the treatment of women with HR-positive, HER2-negative advanced or metastatic breast cancer with disease progression following endocrine therapy.

There is always a need to find new antitumoral treatments. Now, it is shown herein that a combination of compound (1) with abemaciclib is well tolerated, demonstrates significant anti-tumor efficacy, and induces tumor stasis, with a synergistic effect compared to each of the active ingredient alone.

Herein is provided a combination comprising compound (1) and abemaciclib.

In the combination provided herein, compound (1) may exist not only in the form of a zwitterion (i.e. a globally neutral molecule having an acid group and a basic group), but also in the form of addition salts with acids or bases. Such addition salts may be used in the above combination. Hence, herein is provided a combination comprising compound (1), or a pharmaceutically acceptable salt thereof, and abemaciclib.

In an embodiment, the combination of compound (1), or a pharmaceutically acceptable salt thereof, with abemaciclib shows therapeutic synergy. A combination demonstrates therapeutic synergy if its therapeutic effect is superior compared to the cumulative effect of either active agent of the combination alone.

In another embodiment, compound (1), or a pharmaceutically acceptable salt thereof, and abemaciclib are administered by the oral route.

Provided herein is also a combination of compound (1), or a pharmaceutically acceptable salt thereof, and abemaciclib for its use as a medicament.

Provided herein is also a pharmaceutical composition comprising compound (1), or a pharmaceutically acceptable salt thereof, and abemaciclib, as well as at least one pharmaceutically acceptable excipient.

The excipients are selected from the customary excipients which are known to a person skilled in the art. More particularly, the excipients are selected from those useful for oral administration in whatever form (liquid solution, dispersion or suspension, tablets, capsules, or the like).

In another embodiment, compound (1), or a pharmaceutically acceptable salt thereof, and abemaciclib may be administered simultaneously, separately, or spaced out over a period of time (sequential administration). Therefore, the combination and pharmaceutical composition provided herein are not exclusively limited to the ones which are obtained by physical association of the constituents in a single unit dosage, but also to those which allow a separate administration, which can be simultaneous or sequential (also called “spaced out”, or “spread out”) over a period of time.

Herein is also provided a pharmaceutical kit which comprises:
(i) a first pharmaceutical composition comprising compound (1), or a pharmaceutically acceptable salt thereof, and at least one pharmaceutically acceptable excipient;
(ii) a second pharmaceutical composition comprising abemaciclib, and at least one pharmaceutically acceptable excipient;
   wherein the first pharmaceutical composition and the second pharmaceutical composition are in separate compartments and are intended to be independently administered, each administration with regards to the other one being simultaneous or spaced out (sequential) over time.

In the combinations, pharmaceutical compositions and pharmaceutical kit described above, the compound (1) or pharmaceutically acceptable salt thereof and abemaciclib are advantageously present at effective doses, adapted considering the treated pathology and the condition of the patient to which the combination, pharmaceutical composition or pharmaceutical kit is administered. In particular, for abemaciclib the recommended starting dose for adult patients is 200 mg twice daily in monotherapy, and 150 mg twice daily as combination therapy with fulvestrant, taken orally with or without food.

Herein is also provided a combination comprising compound (1), or a pharmaceutically acceptable salt thereof, and abemaciclib, as well as a pharmaceutical composition and kit as described above, for use in the treatment of cancer.

Herein is also provided compound (1) or a pharmaceutically acceptable salt thereof for use in the treatment of cancer by co-administration with abemaciclib.

Herein is also provided abemaciclib for use in the treatment of cancer by co-administration with compound (1) or a pharmaceutically acceptable salt thereof.

Co-administration is understood herein as an administration of the active ingredients to a patient in need thereof, which is separated, simultaneous, or spaced out (sequential) over time, in respect of each of the active ingredient.

In some embodiments, compound (1) or a pharmaceutically acceptable salt thereof and abemaciclib are administered in a therapeutically effective amount. A “therapeutically effective amount” means the amount of an active ingredient or combination of active ingredients that, when administered to a patient for treating a disease, is sufficient to affect such treatment for the disease. The “therapeutically effective amount” will vary depending on the disease and its severity and the age, weight, etc., of the mammal (for example, a human patient) to be treated.

In some embodiments, compound (1) or a pharmaceutically acceptable salt thereof and abemaciclib are administered in an amount to show therapeutic synergy.

In another embodiment, the cancer is a hormone dependent cancer.

In another embodiment, the cancer is an estrogen receptor dependent cancer, particularly the cancer is an estrogen receptor α-dependent cancer.

In another embodiment, the cancer is resistant to anti-hormonal treatment.

In another embodiment, the cancer is a cancer with wild type estrogen receptors.

In another embodiment, the cancer is a cancer with deregulated function of estrogen receptors related to, but not limited to, at least one epigenetic and genetic alteration of estrogen receptors such as mutation, amplification, or splice variant.

In another embodiment, the cancer is a cancer with mutated estrogen receptors.

In another embodiment, the cancer is an estrogen-sensitive cancer.

In another embodiment, the cancer is breast cancer, more particularly an estrogen receptor positive breast cancer (more specifically, an ERα positive breast cancer), or a metastasis thereof, such as a cerebral metastasis.

Herein is also provided a method of treating the pathological conditions indicated above, particularly breast cancer, comprising administering to a patient in need thereof a therapeutically effective amount of compound (1), or a pharmaceutically acceptable salt thereof, and a therapeutically effective amount of abemaciclib.

Herein is also provided a method of treating the pathological conditions indicated above, particularly breast cancer, comprising administering to a patient in need thereof a pharmaceutical composition or a pharmaceutical kit as described above.

Herein is also provided a method of treating the pathological conditions indicated above, particularly breast cancer, comprising administering to a patient in need thereof a combination as described above.

Herein is also provided a method of treating the pathological conditions indicated above, particularly breast cancer, comprising co-administering to a patient in need thereof compound (1) or a pharmaceutically acceptable salt thereof and abemaciclib. In said method, compound (1) or a pharmaceutically acceptable salt thereof is administered with abemaciclib either simultaneously or spaced out over time.

Herein is also provided a method of treating the pathological conditions indicated above, particularly breast cancer, comprising co-administering to a patient in need thereof abemaciclib and compound (1) or a pharmaceutically acceptable salt thereof. In said method, abemaciclib is administered with compound (1), or a pharmaceutically acceptable salt thereof, either simultaneously or spaced out over time.

Herein is also provided a method of treating cancer comprising administering to a patient in need thereof a therapeutically effective amount of compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, in combination with a therapeutically effective amount of abemaciclib.

Herein is also provided a method of treating cancer in a patient who is on therapy with compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, comprising administering to said patient an effective amount of abemaciclib.

Herein is also provided a method of treating cancer in a patient on stable treatment with compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, comprising administering to said patient a therapeutically effective amount of abemaciclib.

Herein is also provided a method of treating cancer comprising administering to a patient in need thereof a therapeutically effective amount of abemaciclib, wherein said patient is also on therapy with compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof.

In an embodiment of the methods described above, the patient is a human patient.

Herein is also provided a combination comprising compound (1), or a pharmaceutically acceptable salt thereof, and abemaciclib for the manufacture of a medicament useful in treating the pathological conditions indicated above, particularly breast cancer.

Herein is also provided the use of compound (1), or a pharmaceutically acceptable salt thereof, in the manufacture of a medicament useful in treating the pathological conditions indicated above, particularly breast cancer, by co-administration with abemaciclib.

Herein is also provided the use of abemaciclib in the manufacture of a medicament useful in treating the pathological conditions indicated above, particularly breast cancer, by co-administration with compound (1) or a pharmaceutically acceptable salt thereof.

Herein is also provided an article of manufacture, a packaging, or an administration unit, comprising:
a packaging material;
the above defined combination, pharmaceutical composition, or pharmaceutical kit; and
a label or package insert contained within said packaging material, indicating that said combination, pharmaceutical composition, or pharmaceutical kit is administered to a patient for the treatment of cancer.

The examples below show the pharmacological results obtained with compound (1), abemaciclib and their combination against a breast cancer cell line xenograft in mice.

### Evaluation of the Efficacy of 6-(2,4-dichlorophenyl)-5-[4-[(3S)-1-(3-fluoropropyl) pyrrolidin-3-yl]oxyphenyl]-8,9-dihydro-7H-benzo[7]annulene-2-carboxylic Acid Combined with Abemaciclib Against a Subcutaneous Breast Cancer Cell Line Xenograft in Female Nude Mice

In the present study, the anti-tumor efficacy of 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid (“compound (1)”), combined with abemaciclib, was investigated after 22 days treatment against a subcutaneous MCF7-Y537S human breast cancer cell line xenograft in female nude mice.

The treated groups included compound (1) at 20 mg/kg alone, abemaciclib at 70 mg/kg alone, and the combination of compound (1) and abemaciclib at the same dose and regime,

Compound (1) was orally dosed twice a day (BID) and abemaciclib was orally dosed once a day (QD) for 22 days. Anti-tumor efficacy was evaluated by tumor volume measurement.

### 1: EXPERIMENTAL PROCEDURE

1-1: Animals, Cell Line, Compounds

Female BALB/c nude mice were obtained from Shanghai Sino-British SIPPR/BK Laboratory Animal Co., LTD (Shanghai, CHINA). Animals were allowed to acclimate for at least four days before the study enrollment. Mice were 6 to 8 weeks old and weighed between 18 and 24 grams at the beginning of the treatments. These animals were housed under conditions outlined in the guidelines approved by the Institutional Animal Care and Use Committee (IACUC) of WuXi AppTec following the guidance of the Association for Assessment and Accreditation of Laboratory Animal Care (AAALAC).

Parental MCF7 cells were obtained from the American Type Culture Collection (ATCC® HTB-22™). MCF7-Y537S (ESR1) cell line was MCF7 cells expressing the ER.Y537S variant that was generated by Sanofi Biology Discovery Group. Y537S mutation was introduced in ESR1 construct (GenBank NM_000125.3) by site directed mutagenesis (Toy W. et al., Cancer Discovery, 2017, 7, 277-287). The construct was transfected in MCF7 cells which were selected for their growth in absence of estradiol. MCF-Y537S is an ESR1 mutation that confers estrogen-independent activity to ERα (Estrogen Receptor alpha) and contributes to endocrine resistant disease (Robinson D. R. et al., Nat Genet., 2013, 45 (12), 1446-1451). The cells were grown in Eagle's Minimum Essential Medium (EMEM) supplemented with 10% fetal bovine serum (FBS), human Insulin, in 5% CO₂ at 37° C. The cells were harvested in 0.25% Trypsin EDTA and washed by Phosphate Buffered Saline (PBS) and re-suspended in PBS with 75% Matrigel. The cells (20×10⁶ cells/per mouse) were subcutaneously (SC) implanted into the right flank of female nude mice.

When the MCF7-Y537S tumors were established, the tumors were reserved as tumor stocks for fragment implantation. The tumors were serially propagated through fragment tissue transplantation subcutaneously. The fragment tumor tissues were subcutaneously implanted into the right flank of female nude mice. 28 mice were assigned in this experiment.

Abemaciclib (Manufacturer: Sanofi; Lot number: VAC.DLE20.006.1) was formulated in 40% SBE-β-CD in HCl 0.1N pH 3.0. Compound (1) was prepared in 5% Solutol HS15 (purchased from Sigma) at pH 3.0.

Dose volume for compound (1) and abemaciclib for oral administration: 10 ml/kg by oral gavage.

Doses: compound (1) at 20 mg/kg and abemaciclib at 70 mg/kg in the above volume.

1-2: Study Design, End Points

The animals required for experiment (plus extra) were pooled and implanted with MCF7-Y537S tumor fragment tissues. On day 0 (20 days post implantation), the mice were pooled and randomly distributed to the treatment and control groups (7 mice per group), where median tumor volumes for each group was 173 mm³. Treatments of compound (1) and abemaciclib were initiated on day 0. Compound (1) was orally administered at 20 mg/kg BID (8 hours apart) and abemaciclib was orally administered at 70 mg/kg QD, for 22 days. Animal body weight was assessed daily.

The dosages are expressed in mg/kg and based on daily body weight per animal. Vehicle treated animals were used as controls. Mice were checked daily and adverse clinical reactions noted. Individual mice were weighed daily until the end of the experiment. Mice would be euthanized when morbid or weight loss ≥20% was observed. Tumors were measured with a caliper twice weekly until final sacrifice. When a tumor size reached approximately 2000 mm³ or when there are animal health issues (40% area of a tumor ulcerated), animals would be euthanized and date of death recorded. Solid tumor volumes were estimated from two-dimensional tumor measurements and calculated according to the following equation:

$$Tumor volume ( {mm}^{3} ) = \frac{len gth ( m m ) \times {width}^{2} ( {mm}^{2} )}{2}$$

Toxicity End Points:

A dosage producing either 15% body weight loss during 3 consecutive days for an individual mouse, 20% body weight loss during 1 day, or 10% or more drug related deaths, was considered an excessively toxic dosage, unless under certain circumstances bodyweight loss or animal death can be considered non-drug related. Examples include animal handling issues such as misgavage, tumor model related issues such as tumor induced cachexia leading to body weight loss that can be observed in control or vehicle treated groups and excessive tumor ulceration. Mice that have non-drug related death or significant bodyweight loss will not be considered toxic and will be excluded from statistical analysis. Animal body weight included the tumor weight.

Efficacy End Points:

The primary efficacy end points include tumor volume changes from baseline summarized by the ratio of medians of tumor volume changes from baseline between the treated and control groups (ΔT/ΔC). Changes in tumor volume for each treated (T) and control (C) group are calculated for each animal on each day by subtracting the tumor volume on the day of first treatment (staging day) from the tumor volume on the specified observation day. The median ΔT is calculated for the treated group and the median ΔC is calculated for the control group. The ratio ΔT/ΔC is calculated and expressed as percentage:

$$Δ T / Δ C = ( \frac{Median delta T}{Median delta C} ) \times 100$$

ΔT/ΔC≤40% is considered as therapeutically active, ΔT/ΔC=0% is considered as tumor stasis, and ΔT/ΔC<0% is considered as tumor regression (very active). ΔT/ΔC>40% is considered as therapeutically inactive.

Percent tumor regression is defined as % (percentage) of tumor volume decrease in the treated group on a specified observation day compared to its volume when the study was initiated. At a specific time point (t) and for each animal, the regression percentage is calculated using the following formula:

$$\% regression ( at t ) = ( \frac{{volume}_{t 0} - {volume}_{t}}{{volume}_{t 0}} ) \times 100$$

The median percent regression for a group on a given day is then calculated by taking the median of individual % regression values calculated for each animal in the group. The day of calculation is determined by the day when ΔT/ΔC is calculated, excepted if median percent regression is not representative of the activity of the group. In this case, the day is determined by the first day when the median percent regression is maximal.

1-3: Statistical Analysis

A two-way Anova-Type analysis with factors treatment and day (repeated) is performed on tumor volume changes from baseline. It is followed by contrast analyses with Bonferroni-Holm correction for multiplicity to compare all treated groups to the control group and to compare the combination versus each single agent at the dose involved in the combination at each day from day 0 to 22.

In the figures, the medians and Median Absolute Deviation (MAD) of each group are represented for each day of measurement.

In the tables, the medians and Normalized MAD (nMAD=1.4826*MAD) of each group are reported for each day of measurement.

Tumor volume changes from baseline are calculated for each animal and each day by subtracting the tumor volume on the day of first treatment (day 0) from the tumor volume on the specified observation day.

All statistical analyses were performed using SAS version 9.2 software. A probability of less than 5% (p<0.05) was considered as significant.

2: Results

Compound (1) at 20 mg/kg BID, abemaciclib 70 mg/kg QD and the combination of compound (1) and abemaciclib at the doses and regime for 22 days were well tolerated and no significant body weight loss was observed in the study.

Compound (1) at a dose of 20 mg/kg BID for 22 days had no statistically significant anti-tumor effect on tumor growth with ΔT/ΔC value of 47% (p=0.9411) on day 22. Abemaciclib at a dose of 70 mg/kg QD for 22 days induced statistically significant anti-tumor efficacy with ΔT/ΔC value of 19% (p=0.0002) on day 22. When compound (1) at 20 mg/kg combined with abemaciclib 70 mg/kg with the same dose regime as BID for compound (1) and QD for abemaciclib, the combination treatment demonstrated statistically significant anti-tumor efficacy (tumor stasis) with ΔT/ΔC value of −4% (p<0.0001) on day 22. The statistical analysis indicated the combination effect was significantly different when compared to either compound (1) alone or abemaciclib alone on day 22 (p<0.0001).

Detailed results are shown in Tables 1 to 3 below, as well as in FIGS. 1 and 2.

### BRIEF DESCRIPTION OF THE DRAWINGS

FIG. 1: Antitumor activity of compound (1) combined with abemaciclib against subcutaneous human breast cancer cell line MCF7-Y537S xenograft in nude mice: tumor volume evolution. The curves represent medians + or − MAD (Median Absolute Deviation) at each day for each group;

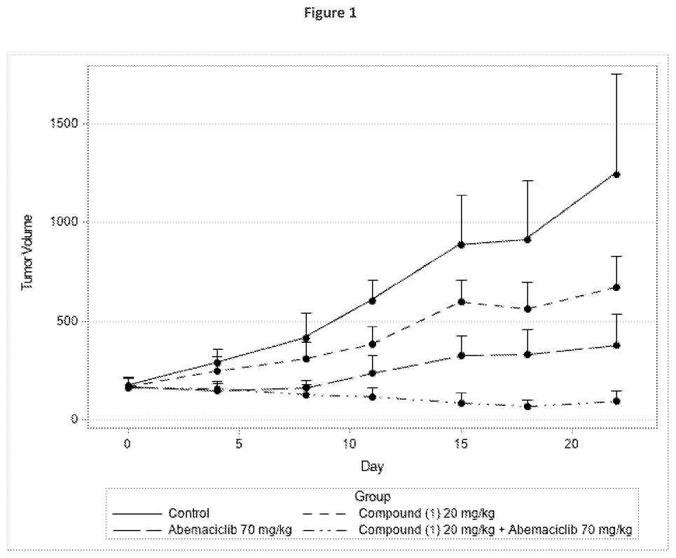

FIG. 2: Antitumor activity of compound (1) combined with abemaciclib against subcutaneous human breast cancer cell line MCF7-Y537S xenograft in nude mice: tumor volume changes from baseline on day 22. Points represent individual tumor volume changes from baseline on day 22, bars correspond to medians.

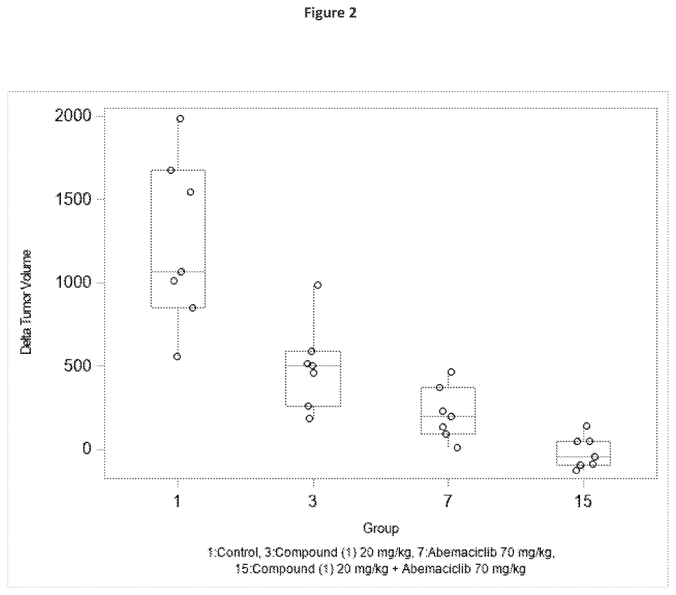

From this experiment, we conclude that compound (1) at 20 mg/kg twice a day combined with the CDK4/6 inhibitor abemaciclib at 70 mg/kg once a day induced significant anti-tumor efficacy in MCF7-Y537S human breast cancer cell line xenograft model in nude mice that was superior to single agents alone, and induced tumor growth inhibition and tumor stasis.

TABLE 1
Efficacy of compound (1) combined with abemaciclib against subcutaneous
MCF7-Y537S human breast cancer xenograft in nude mice. PO: per os

| | Route/ | Dosage | | | | Median % | | | | |
|---|---|---|---|---|---|---|---|---|---|---|
| | Dosage | in | Schedule | *Un- | | of | | | | Bio- |
| | (in mL/kg | mg/kg | in days | scheduled | ΔT/ΔC | regressions | | | p-value | logical |
| | per | per | (total of | death | in % | on | Regressions | | on | Inter- |
| Agent | injection) | injection | 22 days) | (Day of death) | at day 22 | day 22 | Partial | Complete | day 22 | pretation |
| Vehicle | PO, BID (10) | — | — | 0/7 | 100 | — | 0/7 | 0/7 | — | — |
| Compound (1) | PO, BID (10) | 20 | 0 to 22 | 0/7 | 47 | 0 | 0/7 | 0/7 | p = 0.9411 | Inactive |
| Abemaciclib | PO, QD (10) | 70 | 0 to 22 | 0/7 | 19 | 0 | 0/7 | 0/7 | p = 0.0001 | Active |
| Compound | PO, BID (10) | 20 | 0 to 22 | 0/7 | −4 | −10 | 4/7 | 0/7 | p < 0.0001 | Very active |
| (1) + | PO, QD | + | | | | | | | | |
| Abemaciclib | (10) | 70 | | | | | | | | |

TABLE 2
Efficacy of compound (1) combined with abemaciclib against subcutaneous
human breast cancer cell line MCF7-Y537S xenograft model in nude mice.
Comparison of each group to the control group at each day.
Tumor volume changes from baseline mm³:Median (nMAD)*, n and p-value^{#}

| Treatment | | | | | | | |
|---|---|---|---|---|---|---|---|
| Group | Global | Day 4 | Day 8 | Day 11 | Day 15 | Day 18 | Day 22 |
| Control | — | 87.0 | 237.0 | 430.0 | 713.0 | 738.0 | 1065.0 |
| | | (25.20) | (105.26) | (75.61) | (272.80) | (382.51) | (717.58) |
| | | n = 7 | n = 7 | n = 7 | n = 7 | n = 7 | n = 7 |
| Compound | — | 43.0 | 121.0 | 214.0 | 360.0 | 392.0 | 501.0 |
| (1) | | (51.89) | (45.96) | (69.68) | (220.91) | (100.82) | (131.95) |
| 20 mg/kg | | n = 7 | n = 7 | n = 7 | n = 7 | n = 7 | n = 7 |
| | 0.2279 | 0.5181 | 0.2234 | 0.1776 | 0.3604 | 0.4047 | 0.9411 |
| Abemaciclib | — | 9.0 | 40.0 | 83.0 | 119.0 | 150.0 | 199.0 |
| 70 mg/kg | | (41.51) | (50.41) | (114.16) | (91.92) | (149.74) | (160.12) |
| | | n = 7 | n = 7 | n = 7 | n = 7 | n = 7 | n = 7 |
| | <.0001 | 0.0057 | <.0001 | <.0001 | <.0001 | <.0001 | 0.0002 |
| Compound | — | 13.0 | −37.0 | −38.0 | −49.0 | −43.0 | −47.0 |
| (1) | | (51.89) | (85.99) | (63.75) | (109.71) | (127.50) | (118.61) |
| 20 mg/kg + | | n = 7 | n = 7 | n = 7 | n = 7 | n = 7 | n = 7 |
| Abemaciclib | | | | | | | |
| 70 mg/kg | <.0001 | 0.0057 | <.0001 | <.0001 | <.0001 | <.0001 | <.0001 |

^{#}p-values obtained with a contrast analysis versus control at each day with Bonferroni-Holm adjustment for multiplicity after a two-way Anova-Type on tumor volume changes from baseline.
*MAD = Median Absolute Deviation;
nMAD = normalized MAD;
nMAD = 1.4826*MAD.
For the combination compound (1) at 20 mg/kg + Abemaciclib at 70 mg/kg, the effect on tumor volume changes from baseline is significant compared to the control group from day 4 to day 22.
n = number of animals.

TABLE 3
Efficacy of compound (1) combined with abemaciclib against subcutaneous
human breast cancer cell line MCF7-Y537S xenograft model in nude mice.
Comparison of compound (1) 20 mg/kg and abemaciclib 70 mg/kg
as single agents versus the combination at each day.
Tumor volume changes from baseline mm³:Median (nMAD)*, n and p-value^{#}

| Treatment | | | | | | | |
|---|---|---|---|---|---|---|---|
| Group | Global | Day 4 | Day 8 | Day 11 | Day 15 | Day 18 | Day 22 |
| Compound (1) | — | 13.0 | −37.0 | −38.0 | −49.0 | −43.0 | −47.0 |
| 20 mg/kg | | (51.89) | (85.99) | (63.75) | (109.71) | (127.50) | (118.61) |
| + | | n = 7 | n = 7 | n = 7 | n = 7 | n = 7 | n = 7 |
| Abemaciclib | | | | | | | |
| 70 mg/kg | | | | | | | |
| Abemaciclib | — | 9.0 | 40.0 | 83.0 | 119.0 | 150.0 | 199.0 |
| 70 mg/kg | | (41.51) | (50.41) | (114.16) | (91.92) | (149.74) | (160.12) |
| | | n = 7 | n = 7 | n = 7 | n = 7 | n = 7 | n = 7 |
| | 0.0055 | 1.0000 | 0.5471 | 0.0045 | 0.0003 | 0.0001 | <.0001 |
| Compound (1) | — | 43.0 | 121.0 | 214.0 | 360.0 | 392.0 | 501.0 |
| 20 mg/kg | | (51.89) | (45.96) | (69.68) | (220.91) | (100.82) | (131.95) |
| | | n = 7 | n = 7 | n = 7 | n = 7 | n = 7 | n = 7 |
| | <.0001 | 0.5762 | <.0001 | <.0001 | <.0001 | <.0001 | <.0001 |

^{#}p-values obtained with a contrast analysis to compare the combinations of compound (1) and abemaciclib versus each single agent at the dose involved in the combination at each day with Bonferroni-Holm adjustment for multiplicity after a two-way Anova-Type on tumor volume changes from baseline.
*MAD = Median Absolute Deviation;
nMAD = normalized MAD;
nMAD = 1.4826*MAD
The effect of the combination of compound (1) at 20 mg/kg + abemaciclib at 70 mg/kg is significantly greater than the effect of abernaciclib at 70 mg/kg alone on day 4 to day 22.
The effect of the combination of compound (1) at 20 mg/kg + abemaciclib at 70 mg/kg is significantly greater than the effect of compound (1) at 20 mg/kg alone on day 4 to day 22.
n = number of animals.

1. A combination comprising 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, and abemaciclib.

2. The combination according to claim 1, showing therapeutic synergy.

3. The combination according to claim 1 or claim 2, for use in the treatment of cancer.

4. The combination for use according to claim 3, wherein the cancer is breast cancer.

5. The combination according to any of claims 1 to 4, wherein 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, and abemaciclib are administered simultaneously or spaced out over a period of time.

6. A pharmaceutical composition comprising 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, and abemaciclib, and at least one pharmaceutically acceptable excipient.

7. The pharmaceutical composition according to claim 6, for use in the treatment of cancer.

8. The pharmaceutical composition for use according to claim 7, wherein the cancer is breast cancer.

9. Compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof for use in the treatment of cancer by co-administration with abemaciclib.

10. The compound for use in the treatment of cancer according to claim 8, which is administered separately, simultaneously, or spaced out over time, with abemaciclib.

11. Abemaciclib for use in the treatment of cancer by co-administration with compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof.

12. Abernaciclib for use in the treatment of cancer according to claim 11, which is administered separately, simultaneously or spaced out over time, with compound 6(2,4-dichlorophenyl)-5-[4-[(3S)-1-(3-fluoropropyl)pyrrolidin-3-yl]oxyphenyl]-8,9-dihydro-7H-benzo[7]annulene-2- carboxylic acid, or a pharmaceutically acceptable salt thereof.

13. A method of treating cancer comprising administering to a patient in need thereof a therapeutically effective amount of compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, in combination with a therapeutically effective amount of abemaciclib.

14. A method of treating cancer in a patient who is on therapy with compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, comprising administering to said patient an effective amount of abemaciclib.

15. A method of treating cancer in a patient on stable treatment with compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, comprising administering to said patient a therapeutically effective amount of abemaciclib.

16. A method of treating cancer comprising administering to a patient in need thereof a therapeutically effective amount of abemaciclib, wherein said patient is also on therapy with compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof.

17. A pharmaceutical kit comprising:
(i) a first pharmaceutical composition comprising 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or a pharmaceutically acceptable salt thereof, and at least one pharmaceutically acceptable excipient;
(ii) a second pharmaceutical composition comprising abemaciclib, and at least one pharmaceutically acceptable excipient;
wherein the first pharmaceutical composition and the second pharmaceutical composition are in separate compartments and are intended to be independently administered, each administration with regards to the other one being simultaneous or spaced out over time.
